(19) United States
(12) Patent Application Publication
(10) Pub. No.: US 2012/0135442 A1
(43) Pub. Date: May 31, 2012

(54) PROTEIN CRYSTALLIZATION USING MOLECULARLY IMPRINTED POLYMERS

(75) Inventors: Subrayal Medapati Venkt Reddy, Guildford (GB); Naomi Esther Chayen, London (GB)

(73) Assignee: UNIVERSITY OF SURREY, Surrey (GB)

(21) Appl. No.: 13/375,770

(22) PCT Filed: May 12, 2010
(86) PCT No.: PCT/GB2010/050775

§ 371 Date: Feb. 17, 2012

(30) Foreign Application Priority Data
Jun. 3, 2009 (GB) 0909502.7

(51) Int. Cl. C12Q 1/37; C12N 9/36; G01N 33/68; C12Q 1/34; C12N 9/08; C12Q 1/30; C12N 9/76; C07K 14/43

(52) U.S. Cl. 435/23; 435/213; 435/206; 530/370; 435/18; 435/192; 435/27; 436/86

(57) ABSTRACT

The invention relates to a method comprising: providing a molecularly imprinted polymer imprinted with a first peptide or protein; exposing said molecularly imprinted polymer to a supersaturated solution of a second peptide or protein; and forming a nucleus of and/or growing a crystal of said second peptide or protein on said molecularly imprinted polymer.

This application claims priority to United Kingdom application No. 0909502.7, filed Jun. 3, 2009, and PCT/GB2010/050775, with international filing date of May 12, 2010, which are hereby incorporated by reference in their entireties.

### FIELD OF THE INVENTION

The present invention relates to the field of protein crystallization, and to a novel protein crystallisation technique.

### BACKGROUND

For practically all proteins, their structure is key to their function. The determination of structure is therefore an essential part of any research into proteins. The most effective technique for structure determination is X-ray crystallography, but this requires high quality crystals of the proteins.

For some proteins that have been well-studied to date, it is known that crystals can be obtained by reducing the solubility of the protein in concentrated solution, i.e. “salting out”. For instance, myoglobin crystallizes in 3M ammonium sulphate. However, there is no single set of crystallization conditions that applies to all proteins, nor are the required conditions for each protein predictable. See Nature Methods, 2008, 5(2), 147-153. Traditionally, it has been necessary to conduct a whole series of trials for any given protein, in order to determine the essential temperature, pH, protein concentration, precipitant etc. for effective crystallization. Such an empirical approach clearly takes a great deal of time and effort. Indeed, protein crystallization has been described as something of an art form. Whilst high-throughput methods are available to conduct over 1000 screens per protein, this approach is still unsatisfactory.

The Human Genome Project has opened up exciting opportunities for the treatment of disease, and protein crystallisation is vital to its success. This is because it is often not genes themselves that are the targets of potential drugs, but the thousands of proteins encoded by these genes. The current structural genomics/proteomics projects worldwide have set out to determine the structures of 100,000 proteins. In spite of investing considerable funds and effort, however, these projects have had limited success because obtaining crystals, particularly those of high enough quality to allow structure determination, is a major bottleneck to progress.

Crystals grow from supersaturated solutions. The best way to obtain good crystals is to control their conception stage, i.e. the nucleation stage that is the first step that determines the entire crystallization process. During the nucleation stage, protein molecules dissolved in the solvent group together to form aggregates. These aggregates can redissolve, but once they reach a certain size, they form nuclei that are stable; the threshold size is influenced by many physical parameters. Once stable nuclei have been obtained, however, spontaneous crystal growth can occur.

Nucleation can take place spontaneously in the bulk of the solution (“homogeneous nucleation”), provided that the supersaturation level is sufficiently high to overcome the nucleation energy barrier. Below this level, the solution is termed “metastable”, meaning that spontaneous nucleation cannot occur but crystals can still grow from existing nuclei. Nevertheless, even under metastable conditions, heterogeneous nucleation can be induced by addition of a solid material that reduces the energy barrier for nucleation, e.g. a seed crystal or other solid that induces nucleation (“nucleant”).

Various materials have been investigated as nucleants for protein crystallization, including minerals and human hair, but success has been limited. Researchers in this field have been searching for a “universal nucleant”: a substrate that would induce crystallization of any protein. Porous silicon having a range of pore sizes, a small proportion of which are comparable with the size of protein molecules, was shown to be an effective nucleant for a number of proteins tested in J. Mol. Biol., 2001, 312, 591-595. It was speculated that the pores of the correct shape and size confine and concentrate the protein molecules, inducing the formation of nuclei. However, the pore distribution is insufficient to be effective for a wide range of proteins. There are also practical problems with this material: it has to be cut with a scalpel to an appropriate size, which is inconvenient and adversely affects reproducibility of the technique. In addition, it oxidises over time.

Later, mesoporous bioactive CaO—P₂O₅—SiO₂ gel-glass particles with 2-10 nm pore size were used to crystallize seven proteins, including lysozyme, thaumatin and trypsin (PNAS, 2006, 103(3), 597-601). The particles can be picked up using tweezers and inserted into the protein crystallization solution, which is more convenient than for the porous silicon nucleants; nevertheless, there are still difficulties in the automation of this technique. The effectiveness of crystallization using this “bioglass” does not appear to be significantly enhanced either, compared with the porous silicon nucleants.

Recently, gold nanoparticles, which are either uncoated or coated by alkylthiol with CO₂H-terminated groups, have been used as nucleants for lysozyme and ferritin (Cryst. Res. Technol., 2008, 43(6), 588-593). This method also has its limitations. For the uncoated particles, it is thought that Au—S bonds may be formed between thiol groups of methionin of the proteins and gold atoms on the surface of the nanoparticles, so proteins with a low methionin content may have difficulty in crystallizing using this nucleant. For the coated nanoparticles, it is thought that there are electrostatic interactions between the negative carboxy groups on the coated nanoparticles and positive amino acid residues on the protein surface, such as Lys or Arg, so proteins without such positive surface charge may have difficulty in crystallizing using this nucleant.

In light of the foregoing, it is clear that, despite the advances made in this field to date, no entirely universal approach for crystallizing all types of proteins has been established. There is therefore an urgent need for new and improved ways to crystallise proteins and related biomolecules.

### SUMMARY

Accordingly, the present inventors have made the invention defined in the claims.

In a first aspect of the invention, there is provided a method comprising: providing a molecularly imprinted polymer imprinted with a first peptide or protein; exposing said molecularly imprinted polymer to a supersaturated solution of a second peptide or protein; and forming a nucleus of, and/or growing a crystal of, said second peptide or protein on said molecularly imprinted polymer.

In a second aspect, there is provided a method of purifying a protein or peptide, comprising crystallizing said protein or peptide using the method of the first aspect.

In a third aspect, there is provided a crystal obtainable by a method of either the first or second aspect.

In a fourth aspect, there is provided the use of a molecularly imprinted polymer to form a nucleus of, and/or to crystallize, a peptide or protein.

In a fifth aspect, there is provided a method for screening for appropriate protein or peptide crystallization conditions, comprising:

providing a molecularly imprinted polymer imprinted with a first protein or peptide;

conducting pairs of screening trials for a second protein or peptide, each pair relating to a trial under a different one of a plurality of different sets of conditions, one member of each pair including the molecularly imprinted polymer in a solution of the second protein or peptide and the corresponding other member of the pair including no molecularly imprinted polymer; and:

determining the conditions under which nucleation of the second protein or peptide occurs in the trials that include the molecularly imprinted polymer, but under which no nucleation occurs in the corresponding trials including no molecularly imprinted polymer, thereby identifying metastable conditions for the second protein or peptide; and/or

determining the conditions under which nucleation of the second protein or peptide occurs in the trials that include no molecularly imprinted polymer, but under which nucleation occurs at a faster rate in the corresponding trials including the molecularly imprinted polymer, thereby identifying nucleation conditions for the second protein or peptide.

### BRIEF DESCRIPTION OF FIGURES

FIG. 1 illustrates the formation of a molecular imprinted polymer and its capacity to rebind the template;

FIG. 2 illustrates the hanging drop crystallization technique;

FIG. 3 illustrates a protein crystallization phase diagram;

FIG. 4 illustrates trypsin crystallization trials in 2 μl drops in the presence of trypsin-imprinted polymer (T-MIP) and non-imprinted polymer (NIP): (a) trypsin crystals grown in a drop containing T-MIP; and (b) Drop containing NIP at identical conditions. To test for reproducibility, the experiments were performed in quadruplicate: 4/4 MIPs gave crystals and 4/4 NIPs did not; and

FIG. 5 illustrates progression of the formation of trypsin crystals on trypsin-imprinted MIP: (a) phase separation; and (b) crystalline aggregation at the protein-rich droplets (bottom left) and large single crystal.

### DETAILED DESCRIPTION

After studying the previous attempts in this field, the present inventors came to the view that the randomness, i.e. non-specificity, of the existing techniques was hindering their progress. Rather than continue the search for a single universal nucleant, they realised that high quality crystals could be obtained in a reliable manner with a universal technique that employs a series of nucleants, each of which are more closely tailored to the particular biomolecule to be crystallized. Bringing together knowledge from two disparate fields, they discovered the effectiveness of molecularly imprinted polymers in peptide and protein crystallization.

Molecularly imprinted polymers (MIPs) are known in the art in the context of various fields including drug delivery, diagnostics and separations. They are made by polymerisation around a template substance, which template is subsequently removed to leave a cavity that acts as a selective receptor to which the template can re-bind. As discussed in the review article Biosensors and Bioelectronics, 22 (2007) 1131-1137, various different templates have been used to produce MIPs, including small compounds, peptides, whole proteins, and epitopes of proteins.

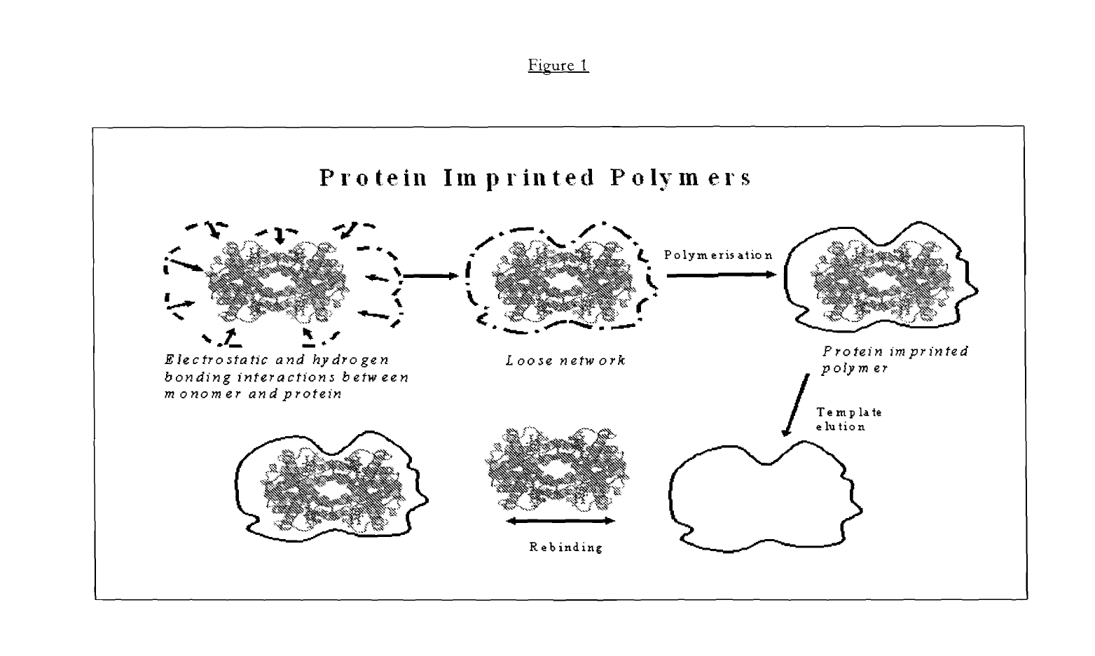

An illustration of the technique using bovine haemoglobin is shown in FIG. 1. When the monomer is mixed with the haemoglobin template in aqueous solution, a loose hydrogen-bonded network is formed between the two. When polymerisation of the loose monomer network is initiated, the protein is preferentially locked and trapped inside a hydrogel matrix due to the attractive intermolecular forces that exist between the protein and the growing polymer. After polymerisation has occurred, the template protein is extracted to leave a cavity that has size, shape and possibly some functional group selectivity for the original protein.

In the present invention, a molecularly imprinted polymer imprinted with a first peptide or protein is used to crystallize a second peptide or protein. The first peptide or protein may be the same as, or different from, the second peptide or protein.

The first and second peptides or proteins may, for instance, each have a molecular weight in the range 1 kDa-100 kDa, 10 kDa-60 kDa or 15 kDa-40 kDa. They may, for instance, have an isoelectric point (pI) in the range 2-15, 3-14, 5-13, 7-12 or 9-11.

When the first and second peptides or proteins are the same, this provides a convenient process in which a biomolecule in solution can be imprinted to form a MIP and then crystallized directly using that same MIP. On the other hand, it is especially convenient for the first and second peptides or proteins to be different where the one to be crystallized is in too scarce a supply to be imprinted itself, or is not easily available in a sufficiently purified form for imprinting.

In an embodiment, the nucleation and/or crystallization stage is improved by increasing the similarity in nature of the first and second peptides or proteins, for instance their size-compatibility. In an embodiment, the molecular weights of the proteins or peptides are within a 20000 Da range, preferably a 15000 Da, 10000 Da, 7000 Da, 5000 Da, 4000 Da, 3000 Da, 2500 Da, 2000 Da, 1000 Da, 500 Da, 100 Da, 50 Da, 20 Da or 5 Da range. In another embodiment, the molecular weight of the second peptide or protein is within a range of ±50% of the molecular weight of the first peptide or protein, preferably ±30%, ±25%, ±20%, ±15%, ±10%, ±5%, ±2% or ±1%.

The second protein or peptide may have a lower molecular weight than the first protein or peptide. In this case, the second protein or peptide may be accommodated by a cavity formed by a single molecule of the first protein or peptide. Alternatively, the second protein or peptide may have a higher molecular weight than the first protein or peptide. Without wishing to be limited by theory, it is thought that in some cases the actual imprinting entity may be an aggregate of molecules of the first protein or peptide, in which case the resulting cavity is of a sufficient size to accommodate the larger second protein or peptide molecule. In other cases, only a part of the second protein or peptide is accommodated in the MIP cavity, but this is still sufficient for nucleation and/or crystallization.

In an embodiment, the first protein or peptide corresponds to an isolated fragment of the second protein or peptide, e.g. an isolated epitope thereof. For instance, if the protein to be crystallized is an immunoglobulin G, in some embodiments the MIP may be imprinted with an isolated Fab or Fc fragment of that immunoglobulin G.

The relative shape of the first and second peptides or proteins may also be important. For instance, both biomolecules could have: an elongated or cylindrical shape (like collagen or tobacco mosaic virus); a round shape (like haemoglobin); a disc shape etc.

In some cases, the relative chemical nature of the first and second peptides or proteins may have an influence. In an embodiment, the first and second peptides or proteins have a pI within a 5 unit range, preferably a 4, 3, 2 or 1 unit range. The first and second peptides or proteins may, for instance, have at least 50%, 60%, 70%, 80%, 90%, 95%, 97%, 98% or 99% sequence identity.

In the present application, the percentage identity between two amino acid (i.e. peptide) sequences is determined in accordance with the skilled person's general knowledge, specifically an alignment of the two sequences is first prepared, followed by calculation of the sequence identity value.

The skilled person will appreciate that the percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants. The skilled person will also appreciate that, having made the alignment, there are several different ways of calculating percentage identity between the two peptide sequences. For example, one may divide the number of identities by: (i) the length of shortest sequence; (ii) the length of alignment; (iii) the mean length of sequence; (iv) the number of non-gap positions; or (iv) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, whilst it will be understood that the accurate alignment of protein sequences is a complex process, the skilled person will be able to select appropriate methods for determining sequence identity from their experience. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins in accordance with the invention. Suitable parameters for ClustalW may be as follows: Gap Open Penalty=10.0, Gap Extension Penalty=0.2, and Matrix=Gonnet; ENDGAP=−1, and GAPDIST=4. Those skilled in the art will be aware that it may be necessary to vary these and other parameters for optimal sequence alignment.

Preferably, calculation of percentage identities between two amino acid/polypeptide sequences is then calculated from such an alignment as (N/T)*100, where N is the number of positions at which the sequences share an identical residue, and T is the total number of positions compared including gaps but excluding overhangs. Hence, a most preferred method for calculating percentage identity between two sequences comprises (i) preparing a sequence alignment using the ClustalW program using a suitable set of parameters, for example, as set out above; and (ii) inserting the values of N and T into the following formula: Sequence Identity=(N/T)*100.

Both peptides or proteins can be water-soluble or non water-soluble, e.g. membrane proteins. Non water-soluble biomolecules can be solubilised in aqueous media by well known techniques, e.g. using a surfactant. In an embodiment, the first and second peptides or proteins have the same solubility properties, i.e. are both water-soluble, or are both non water-soluble.

Examples of the first peptide or protein include: lysozyme, trypsin, thaumatin, catalase, albumin, and cytochrome C; cardiac markers such as myoglobin, troponin, and creatine kinase; and cancer markers such as prostate-specific antigen and alpha fetoprotein.

Imprinting of the first peptide or protein can be conducted using any suitable method. Both solid state and solution-based imprinting strategies are known in the art. For instance, Analyst, 2006, 131, 1044-1050, describes imprinting of a polymer surface with trypsin as the template dissolved in aqueous solution, or in the solid phase (amorphous or crystalline trypsin). Another representative solution-based imprinting technique is described in Analytica Chimica Acta, 2005, 542(1), 61-65.

In the present invention, therefore, crystals of the template can be prepared on “stamps” which are inserted into the polymerising solution. In an embodiment, these crystals are small crystals or “seeds” of the template. Imprinting using pre-formed crystals of the template can be especially useful where the protein or peptide to be crystallized is different from the template. For instance, if a standard crystallization process for a known protein or peptide is well-established, crystals of this biomolecule can be imprinted in the solid phase to form an MIP for crystallization of a related, but previously uncrystallized, protein or peptide.

Alternatively, the template is dissolved or dispersed in a liquid phase and brought into contact with the forming polymer. The molecularly imprinted polymer may be imprinted with the first peptide or protein in the solution phase. Preferably, the template is dissolved in solution. The liquid phase may be aqueous or organic, but is preferably aqueous. The liquid phase may be water, or a miscible combination of water and an organic liquid, e.g. a water/ethanol mixture. It is not essential for the conditions (pH, temperature etc.) to be such as to avoid denaturing of the protein during imprinting. However, in an embodiment, the conditions cause no or only partial denaturing of the protein.

The MIP is preferably an organic polymer, preferably one bearing hydrophilic groups such as hydroxyl, ether, carbonyl, carboxylic acid, ester, or amide groups. In an embodiment of the methods discussed above (wherein the template is in a solid or liquid phase), the polymerising solution comprises a water-soluble monomer component. Preferably, the monomer component is comprised of one or more of the following monomers: an α,β-unsaturated carbonyl compound, such as an α,β-unsaturated acid, ester or amide, or an α,β-unsaturated nitrile. More preferably, the monomer(s) are selected from: acrylamide, an α-alkyl-acrylamide (e.g. methacrylamide), an N-substituted- or N,N-disubstituted-acrylamide, an N-substituted- or N,N-disubstituted-α-alkyl-acrylamide, acrylic acid, an α-alkylacrylic acid (e.g. methacrylic acid), an acrylate, or an α-alkylacrylate (e.g. a methacrylate).

For example, the monomer(s) may be of the formula:

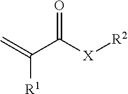

wherein R¹ is H or alkyl; X is O or NR³; R³ is H or alkyl; and R² is H, alkyl, aryl, alkaryl or arylalkyl, each of which may be optionally substituted by one or more hydroxyl, ether, carboxylic acid or ester, amine, amide, imide, sulphonic acid or ester, halogen, or nitrile groups. R¹ and R³ are independently preferably H or C₁₋₅ alkyl, preferably H, methyl, ethyl or propyl, preferably H or methyl. In an embodiment, R² is an optionally substituted alkyl group, preferably an optionally substituted C₁₋₅ alkyl group, the substituent preferably being hydroxyl. The alkyl group may be straight chain or branched; for instance, R² may be ethyl, n-propyl, isopropyl, 2-hydroxyethyl or 2-hydroxypropyl. Examples of the monomer(s) accordingly include 2-hydroxyethylmethacrylate and N-isopropylacrylamide.

In an embodiment, the monomer component comprises (α-alkyl)acrylamide (optionally N-substituted- or N,N-disubstituted) and (α-alkyl)acrylic acid. Preferably, it comprises (meth)acrylamide (optionally N-substituted- or N,N-disubstituted) and (meth)acrylic acid. Preferably, it comprises acrylamide and methacrylic acid.

Polymerisation may be initiated by any suitable means, including by free radical initiation and/or UV irradiation. In an embodiment, it is initiated by free radical initiation, preferably using a persulphate, preferably ammonium persulphate, preferably used in conjunction with N,N,N′,N′-tetramethylethyldiamine (TMEDA) as a catalyst. This embodiment is advantageously used where the monomer component comprises acrylamide-based monomer(s). In another embodiment, it is initiated by UV irradiation in the absence of oxygen, preferably using azobisisobutyronitrile (AIBN) (advantageously where the monomer component comprises acrylate-based monomer(s)) or riboflavin (advantageously where the monomer component comprises acrylamide-based monomer(s)).

The polymer may be cross-linked using a cross-linking component comprised of one or more cross-linking agents. Suitable cross-linking agents are generally compounds having two terminal double bonds, e.g. two [symbol] C═CH₂ groups. In an embodiment, the cross-linking agent is a di(meth)acrylate, di(meth)acrylamide or dialkenyl compound such as a divinyl compound. Examples are: N,N′-alkylene-bis(meth)acrylamides such as N,N′-methylene-, -ethylene-, -propylene-, -tetramethylene-, -pentamethylene- and -hexamethylene-bis(meth)acrylamide; ethylene glycol di(meth)acrylate, 1,4,3,6-dianhydro-d-sorbitol-2-5-di(meth)acrylate, isopropylenebis(1,4-phenylene)di(meth)acrylate, and anhydroerythritol di(meth)acrylate; p-divinylbenzene, and 1,3-diisopropenylbenzene.

Preferably, the molar ratio of the monomer component to the cross-linking component is in the range 10:1 to 30:1, 15:1 to 25:1, or 18:1 to 22:1. Alternatively, the weight of the cross-linking component may be 1-30%, preferably 2-20%, 5-15% or 8-12% of the total weight of the monomer and cross-linking components.

Whichever imprinting method is used, it is in some cases preferred to create a relatively high concentration of cavities in the MIP, since this reduces the likelihood that the cavities are too isolated and inaccessible during the subsequent crystallization process. For instance, a high density of template crystals can be used on the “stamps”, or a high template to monomer ratio in solution can be used (e.g. an excess of the first protein or peptide). However, the concentration of cavities can be limited in order to prevent the formation of too many nuclei, and thereby encourage the growth of a smaller number of large crystals (rather than many small ones). The latter is advantageous if the MIP is to be used to grow crystals for structure determination, but is less important if the MIP is to be used in screening for the metastable state (in which it is only essential to form nuclei).

In an embodiment, the molar ratio of the monomer component to the first protein or peptide in the polymerising solution is in the range 100:1 to 10000:1, 200:1 to 8000:1, 500:1 to 7000:1, 700:1 to 6000:1, or 1000:1 to 5000:1.

The MIP may be produced in any suitable form, including microparticles, as a gel or as a thin film. In an embodiment, it is a gel, preferably a hydrogel (HydroMIP) i.e. a three-dimensional polymer network bearing primarily water in between the polymer chains. Preferably, the polymer chains are held together by covalent bonds, rather than by other means (such as by electrostatic forces, hydrogen bonds, hydrophobic interactions or chain entanglements).

MIPs in gel form are preferably granulated, e.g. by passing through a sieve, to gain access to the template, followed by a template removal step. Template can be removed by any suitable means, including with a digestive enzyme such as trypsin, or by denaturing. In an embodiment, template is removed by washing with a mixture of sodium dodecylsulphate (SDS) and acetic acid (AcOH). The ratio of SDS (w/v):AcOH (v/v) may be, for instance, in the range 1%:1% to 20%:20%, 3%:3% to 17%:17%, 5%:5% to 15%:15% or 8%:8% to 12%:12%.

It should be noted that it is not essential for all the template molecules to be removed; some may remain in the final MIP, in whole or fragmented form, provided that at least some template cavities are present.

The inventors have demonstrated that the method of the invention is not limited to the use of only water-soluble organic monomers for seeding and growing protein crystals, since inorganic MIPs can also be used. Hence, the molecularly imprinted polymer may be an inorganic polymer. In one embodiment, inorganic MIPs (preferably, sol-gel MIPs) may be prepared from a silicon-based monomer (i.e. not carbon-based, as with the organic monomers). Silicon-based HydroMIPs may be prepared using γ-aminopropyl triethoxysilane (APES) as a functional monomer. Tetraethoxysilane (TEOS) may be used as a suitable cross-linker. Hydrochloric acid may be used as a catalyst, and water may be used as the main solvent and ethanol as a co-solvent.

In the preparation of the MIP, the template protein may be added to the APES solution prior to mixing with the TEOS solution. Once the solutions have been mixed together in the presence of the catalyst, polymerization occurs to form a polysiloxane scaffold gel structure around the template protein molecules. The gel may then be ground down, and the protein may be washed away, for example using SDS/acetic acid (10%/10%) solution. Molecularly imprinted cavities, which were selective for the rebinding of the template protein, are then left in the inorganic gel. These protein selective cavities may be used for the seeding and selective nucleation and crystallization of the template protein.

In one embodiment, the MIP may be in a sol-gel form. The term “sol” will be known to the skilled technician and can mean a colloid that has a continuous liquid phase in which a solid is suspended in a liquid.

In another embodiment, the MIP is in thin film form. The thin film may, for instance, have a thickness of 200-3000 nm, preferably 300-2000 nm. One advantage of using thin films is that a lower amount of protein is required, both for the imprinting and the crystallizing stage. Another advantage for growth of a small number of high-quality crystals is that the number of exposed cavities is reduced. In addition, the processing step of passing the MIP through a sieve to release its template can be avoided.

Thin films can be made, for instance, by compression moulding i.e. applying pressure between solid surfaces during gel formation. Alternatively, the polymerising solution can be spin coated onto a surface. In an embodiment, the thin film MIP is sufficiently viscous/gelatinous to be pipetted.

Once the MIP has been obtained, crystallization of the second protein or peptide can be carried out by any suitable method, but additionally including the MIP in the supersaturated solution of the second protein or peptide. Several conventional techniques are known to the skilled person, such as vapour diffusion methods like the so-called “hanging-drop” and “sitting drop” methods, dialysis, and batch or microbatch techniques.

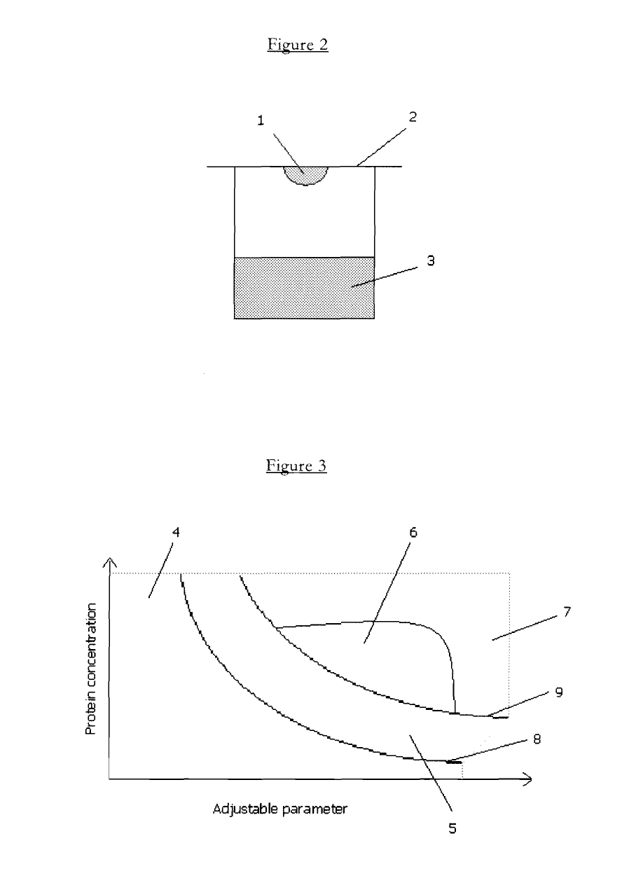

For instance, a hanging-drop crystallization technique is illustrated in FIG. 2. Typically, a small volume of protein solution is mixed with a similar volume of precipitating solution, and a droplet (1) of the mixture is placed on a siliconized glass or plastic cover-slip (2). The cover-slip is inverted and placed over a reservoir of a much larger volume of the crystallizing solution (3). Diffusion through the vapour phase gradually equilibrates the reservoir and the droplet; the protein solution reaches supersaturation, and crystals form.

In the present invention, the MIP can be included in the liquid containing the second protein or peptide, e.g. the drop of the vapour diffusion or microbatch crystallization processes. Any suitable amount of the MIP can be included, e.g. up to 20%, up to 15%, up to 10%, or 1-5%, by volume of the drop.

FIG. 3 is a schematic illustration of a protein crystallization phase diagram, in which the adjustable parameter may be one of various parameters including pH, temperature or precipitant concentration. Area (4) is the undersaturation zone, (5) the metastable zone, (6) the nucleation zone and (7) the precipitation zone. The latter three zones make up the supersaturation region. Curve (8) dividing the metastable and undersaturation zones is termed the solubility curve, whereas curve (9) dividing the metastable and nucleation zones is termed the supersolubility curve.

As discussed at the outset, crystallization is possible in the supersaturated region, but not possible in the undersaturated region. The best conditions for growth of high quality crystals are found in the metastable zone: the region in which spontaneous nucleation cannot occur without added nucleants, but crystals can still grow from existing nuclei. Metastable conditions for any given protein are known or can be determined experimentally by known methods, conducted manually or using robotics. However, the effort required in this initial screening stage can be reduced using the present invention.

When dealing with previously uncrystallized proteins, the chance of forming nuclei is increased by including an MIP nucleant compared with using no nucleant (when nucleation is limited to the nucleation zone). One can conduct a paired series of experiments, each pair relating to a nucleation experiment under a different set of conditions (temperature, pH, precipitant, precipitant concentration etc.), with one member of the pair including the appropriate MIP and the other member having no MIP. By determining the conditions under which nuclei form using a MIP but none form under corresponding conditions without the MIP, metastable conditions can be identified. In some cases, nucleation conditions may also be identified by determining the conditions under which nuclei form more quickly using the MIP.

For this screening method, it is not necessary to grow full crystals. Nucleation can be detected by dynamic light scattering, or resulting tiny crystals or crystalline precipitate can be observed by conventional spectroscopic methods, for instance. Accordingly, one embodiment of the invention involves forming nuclei or any form of crystals of the second peptide or protein on the MIP, but not growing high quality crystals.

Once the metastable conditions have been identified, this information can be used in refining the process. This crystallization optimization stage is also improved using the present invention, because the MIP can afford crystals more reliably where others have struggled to form crystals at all, or may afford crystals of higher quality (e.g. more ordered crystals), as evidenced in the Examples.

Without wishing to be limited by theory, it is thought possible that, when nucleation occurs in the nucleation zone using a MIP, nuclei may be formed spontaneously in solution and then enter the cavities of the MIP, whereupon crystal growth occurs from these MIP-associated nuclei. Accordingly, an embodiment of the invention involves growing crystals of the second peptide or protein on the MIP, but not forming nuclei on the MIP. However, in a preferred embodiment, both nucleation and crystallization occurs on the MIP.

Preferably, nucleation and growth of crystals is conducted under metastable conditions. In an embodiment, the MIP is added at conditions just below the super-solubility curve. This region is easier to identify for the skilled person, and reduces the chance of error in accidentally working in the undersaturated region.

It will be clear to the skilled person that, as well as being a route to structure determination, the present invention is also useful to purify proteins and peptides via their crystallization from an impure solution. Furthermore, known nucleants are solid and therefore not easily amenable to automated dispensing using the liquid handling robots that have become routine in the crystallization laboratory. In contrast, being gels, the MIPs of the invention allow trials to be dispended by these robots thus rendering them highly suitable for high throughput crystallization experiments. Thus, the molecularly imprinted polymer may be used in a high-throughput or automated mode.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention and to show how embodiments of the same may be carried into effect, reference will now be made, by way of example, to the accompanying drawings, in which:—

### EXAMPLES

The invention will now be illustrated by way of the following examples. Trypsin, lysozyme and thaumatin are proteins that have been previously crystallized by standard methods, so these were taken as the initial models for the MIP experiments.

### Example 1

### Preparation of Bulk HydroMIPs

54 mg of monomer (acrylamide), 6 mg of crosslinker (N,N′-methylenebisacryl-amide), and 12 mg of template protein (trypsin) were each dissolved in 1 ml deionized water and the solutions combined to form Solution X. 10 μl/ml of a 10% (w/v) ammonium persulfate (APS) aqueous solution was added to Solution X, which was then purged with nitrogen for 5 minutes. After degassing, 20 μl/ml of a 5% (v/v) N,N,N′,N′-tetramethylethylenediamine (TEMED) aqueous solution was added to form Solution Y, which was then left to polymerize overnight at room temperature.

After polymerization, the gel was crushed using a 75 μm sieve. The crushed gel was transferred into 1.5 ml centrifuge eppendorf tubes, and washed using five 1 ml volumes of RO water and five 1 ml volumes of 10% acetic acid:10% SDS eluant to extract the trypsin. Each wash step was carried out by centrifugation for 3 minutes at 5500 rpm and the supernatants were collected. The gel was then centrifuged for a further 3 minutes at 5500 rpm in order to extract further supernatants.

The resulting trypsin HydroMIP gel is viscous, but can be pipetted.

This same procedure was used to form a hen-egg-white lysozyme-imprinted HydroMIP gel, using 12 mg lysozyme in place of the trypsin.

### Example 2

### Preparation of Control Gel

A non-imprinted polymer (NIP) was prepared by the same method as Example 1, except that no template protein was used.

### Example 3

### Crystallization of Trypsin Using Bulk Trypsin HydroMIP

Crystallization experiments were performed using the hanging drop vapour diffusion method, with Linbro® plates.

A solution of 30 mg/ml trypsin in 100 mM Tris at pH 8.4 was mixed with a 30% ammonium sulphate solution (these are sufficiently metastable conditions for the crystallization of trypsin, i.e. substantially no crystallization will take place unless a suitable nucleant is present). 2 μl crystallization drops were formed, and 0.2-0.5 μl trypsin HydroMIP gel from Example 1 was added to each drop, but not mixed in. The drops were incubated at 20° C. for two weeks. This procedure was repeated for the NIP of Example 2, and for a control using no polymer at all.

Trypsin crystals grew after 3-4 days in each of the drops containing the trypsin HydroMIP, but not in any of the drops containing the NIP at identical times, nor in the other control drops.

### Example 4

### Crystallization of Lysozyme Using Bulk Lysozyme HydroMIP

Example 3 was repeated using the lysozyme HydroMIP of Example 1 in place of the trypsin HydroMIP, and using the following metastable conditions for lysozyme: a solution of 20 mg/ml lysozyme in 20 mM sodium acetate buffer at pH 4.6, mixed with a 2.8% sodium chloride solution. This procedure was repeated for the NIP of Example 2, and for a control using no polymer at all.

Tetragonal lysozyme crystals grew after 4 days in each of the drops containing the lysozyme HydroMIP, but not in any of the drops containing the NIP at identical times, nor in the other control drops.

### Example 5

### Crystallization of Proteins Different from the Ones Imprinted

Examples 3 and 4 were repeated using a number of other proteins instead of trypsin/lysozyme as the protein to be crystallized, under their respective metastable conditions, as detailed below.

Thaumatin: a solution of 30 mg/ml thaumatin in 50 mM PIPES buffer at pH 6.8, mixed with 0.8 M sodium potassium tartrate.

Catalase: a solution of 11 mg/ml catalase in 100 mM Tris at pH 7.5, mixed with 6.5% polyethylene glycol 6000.

The trypsin HydroMIP induced crystallization of lysozyme (crystals after 4 days) and thaumatin (crystals after 7 days). On the other hand, the lysozyme HydroMIP did not induce crystallization of trypsin, thaumatin or catalase. In all cases, no crystals were seen with the NIP of Example 2, or with the control containing no polymer.

Lysozyme has a molecular weight of 14,500 Da, whereas trypsin has a molecular weight of 24,000 Da, thaumatin has a molecular weight of 22,000 Da and catalase has a molecular weight of 232,000 Da. Crystallization of trypsin, thaumatin and catalase using the lysozyme-imprinted MIP may have failed because the lysozyme cavities were too small to accommodate these larger proteins.

On the other hand, the trypsin-imprinted MIP may have had cavities sufficient in size to accommodate the smaller proteins lysozyme and thaumatin, but not the much larger catalase. Although the difference in molecular weight between lysozyme and trypsin is relatively large (lysozyme is 10,500 Da or 44% smaller), lysozyme is a protein that crystallizes very easily, so it is understandable that this would be less sensitive to selectivity requirements.

Thaumatin does not generally crystallize as easily as lysozyme, but this example shows that it can be crystallized under metastable conditions using a MIP imprinted with trypsin; thaumatin is 2,000 Da (or 8%) smaller. In addition, thaumatin and trypsin can crystallize in the same space group in which two of their cell dimensions are almost identical.

This example shows that the MIP of one protein can be successfully used to crystallize another compatible protein.

### Example 6

### Effect of MIPs on Speed of Nucleation Under Nucleation Conditions

Lysozyme, trypsin, thaumatin and catalase were crystallized under their predetermined nucleation conditions, without the use of any added nucleant. In each case, crystals appeared after several days.

The experiments were repeated with added MIPs as follows:

Lysozyme: added lysozyme-imprinted or trypsin-imprinted MIP (both shown to induce nucleation under metastable conditions)
Trypsin: added trypsin-imprinted MIP (shown to induce nucleation under metastable conditions)
Thaumatin: added trypsin-imprinted MIP (shown to induce nucleation under metastable conditions)
Catalase: added lysozyme-imprinted or trypsin-imprinted MIP

Lysozyme, trypsin and thaumatin crystals appeared overnight, i.e. the MIPs that induce nucleation under metastable conditions will speed up nucleation under nucleation conditions. This shows the advantageous effect of using MIPs in a screening process, in which the conditions required to reach the metastable and nucleation states have not yet been identified. As expected, catalase crystals did not appear any faster under nucleation conditions, when using MIPs that do not induce nucleation under metastable conditions.

### Example 7

### Effect of MIPs on Crystal Quality

The trypsin and thaumatin crystals obtained in Example 6 were examined using X-ray beams (at Diamond Light Source). The resolutions obtained were as follows:

Trypsin (made without MIP)—2.3 Å (with MIP)—1.5 Å
Thaumatin (made without MIP)—1.9 Å (with MIP)—1.5 Å

The lower values obtained for the crystals made using a MIP indicates that these crystals were more highly ordered.

### Example 8

### Preparation of Thin Film Trypsin HydroMIP

Solution Y was prepared in the same manner as in Example 1. 25 μl of this solution was applied to a glass slide and covered using a glass cover-slip. 2 Pa of pressure was applied to the solution and it was left to polymerise for 15 min, leaving a film thickness of approximately 20-50 μm.

Once polymerised, the film was washed and eluted by immersing in deionised water and 10% acetic acid:10% SDS eluant for 10 min. The film was then immersed in deionised water in 30 min intervals until it was sufficiently equilibrated.

### Example 9

### Preparation of Control Film

A non-imprinted polymer film was prepared by the same method as Example 3, except that no trypsin was used.

### Example 10

### Crystallization of Further Proteins Different from the Ones Imprinted

MIPs were imprinted with 2 proteins, namely lysozyme and trypsin, and they were tested for their nucleation-inducing properties on 7 proteins. These were their own corresponding proteins i.e. lysozyme and trypsin, and also thaumatin.

0.2 μl of MIP (as a viscous gel) were inserted into 1-2 μl crystallization drops. The same polymer, but not imprinted with protein (i.e. NIP), was also dispensed at the same conditions, for comparison. For each of the proteins tested, a range of conditions were tried, spanning a supersaturation range from those conditions at which crystals would spontaneously form, even in the absence of a nucleation-inducing substance (labile conditions), to those where no crystals would appear even in the presence of a nucleation-inducing substance (undersaturated). The aim was to observe if there were conditions intermediary between these two (metastable), where crystals would only appear in the presence of our polymers.

Table 1 shows the results of the performed experiments at metastable conditions

TABLE 1
Crystallisation results at metastable conditions

| | M.W. | | | | |
|---|---|---|---|---|---|
| Protein | (kDa) | L-MIP | T-MIPS | NIPS | Controls |
| Lysozyme | 14.5 | Crystals | crystals | clear | Clear |
| Thaumatin | 22 | clear | crystals | clear | Clear |
| Trypsin | 24 | Clear | crystals | clear | Clear |

As shown in Table 1, the L-MIP promoted the crystallization of its cognate protein lysozyme. The T-MIP promoted the crystallization of all three proteins. These results indicate that a MIP of one protein can be successfully used as a nucleant for other, size-compatible proteins which is important in the case of difficult to crystallise proteins, that are very scarce in supply.

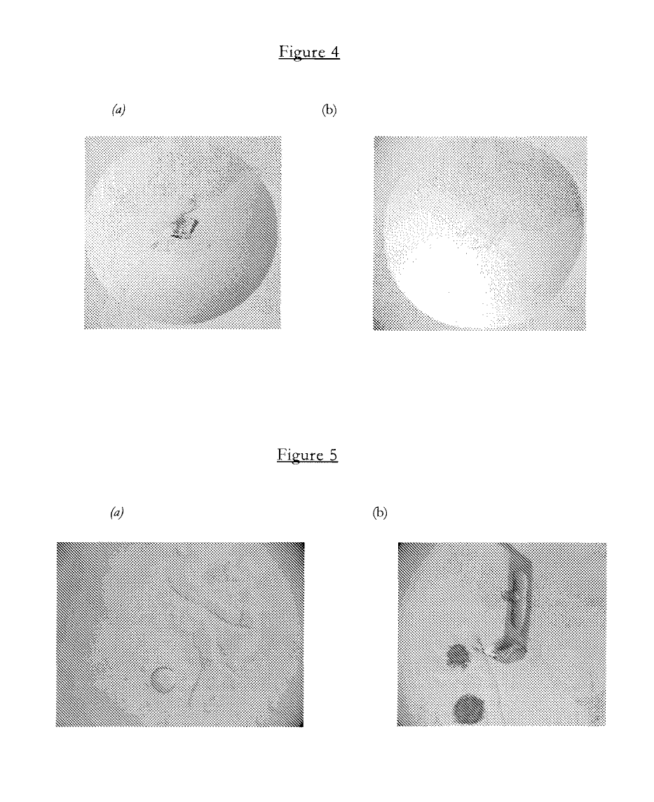

Referring to FIG. 4, there are shown the effects of trypsin crystallization trials in the presence of trypsin-imprinted polymer (T-MIP) and the non-imprinted polymer (NIP). In FIG. 4(a), trypsin crystals are shown growing in a drop containing T-MIP. In FIG. 4 (b), no trypsin crystals formed in a drop containing NIP at identical conditions. At lower supersaturations, all drops remained clear for at least several weeks whereas at higher supersaturations all conditions gave crystals with the crystals forming faster in the presence of the MIPs.

Referring to FIG. 5, there is shown the formation of trypsin crystals on trypsin-imprinted MIP. FIG. 5(a) shows phase separation, and FIG. 5 (b) shows crystalline aggregation at the protein-rich droplets (bottom left) and a large single crystal. It is interesting to note how the crystals often evolve from the MIP: (i) first a separation of liquid phases occurs, forming protein-rich droplets on the MIP (see FIG. 5a); (ii) crystalline aggregation is later observed in these droplets (FIG. 5b, bottom left); (iii) single, large and well-diffracting crystals appear from these protein-rich areas (FIG. 5b). It has been shown theoretically that protein crystal nucleation may proceed in two steps, namely aggregation of molecules and then ordering. Instead of a single, steep energy barrier which would occur if ordering of the molecules happened at the same time as their aggregation, the inventors believe that there would be two lower barriers if the two processes happened separately and in succession. It seems that the MIPs, very soon after their insertion (overnight for lysozyme and trypsin) promote aggregation of protein molecules to form a protein-rich phase, which at a later stage becomes crystalline.

MIPs have an additional important advantage over all other nucleants known to date which are solid and therefore not easily amenable to automated dispensing using the liquid handling robots that have become routine in the crystallization laboratory. Being gels, MIPs enable trials to be dispended by these robots thus rendering them suitable for high throughput crystallization experiments. Consequently MIPs can also be effective in screening trials when searching for initial potential crystallization conditions, the very crucial first step of crystallizing proteins.

The results presented are relevant to two different fields, namely protein crystallization, and, development of biosensors for proteins of medical interest. From the crystallization aspect, the findings open up a new scope for protein crystallization corroborating the inventors' hypothesis that by harnessing the proteins themselves as templates, MIPs are effective nucleation inducing substrates. From the biosensors aspect, the data revealing that lysozyme or trypsin MIP can be used for crystallization of other proteins, has important implications for the research and economical large-scale production of MIP based biosensors for the detection of proteins that are often expensive, difficult to isolate, and possibly hazardous. Alternative MIPs, imprinted for example with lysozyme or trypsin, could be used as safer and cheaper surrogate MIPs for biosensor detection of such proteins.

### Example 11

### Use of Inorganic Sol-Gel MIPs

As described in the previous examples, the inventors have demonstrated that water-soluble organic monomers can be effectively used for growing protein crystals. The inventors have also shown that inorganic MIPs can be used to form a nucleus of (i.e. seeding) and/or growing crystals of proteins. Hence, inorganic sol-gels are another category of hydroMIPs, defined by the invention. The HydroMIPs discussed in the previous examples are prepared from water-soluble organic monomers. However, inorganic sol-gel MIPs were prepared from silicon-based monomers (i.e. not carbon-based, as with the organic monomers).

The silicon-based HydroMIPs were prepared using γ-aminopropyl triethoxysilane (APES) as a functional monomer, and tetraethoxysilane (TEOS) as a cross-linker Hydrochloric acid was used as a catalyst, and water was the main solvent and ethanol was a co-solvent. In the preparation of the MIP, the template protein was added to the APES solution prior to mixing with the TEOS solution. Once the solutions were mixed together in the presence of the catalyst, polymerization occurred to form a polysiloxane scaffold gel structure around the template protein molecules.

Upon grinding of the gel, the protein was then washed away using SDS/acetic acid (10%/10%) solution. Molecularly imprinted cavities, which were selective for the rebinding of the template protein, were then left in the inorganic gel. These protein selective cavities could be used for the seeding and selective nucleation and crystallization of the template protein.

1. A method comprising: providing a molecularly imprinted polymer imprinted with a first peptide or protein; exposing said molecularly imprinted polymer to a supersaturated solution of a second peptide or protein; and forming a nucleus of, and/or growing a crystal of, said second peptide or protein on said molecularly imprinted polymer.

2. A method as claimed in claim 1, wherein the first peptide or protein is the same as the second peptide or protein.

3. A method as claimed in claim 1, wherein the first peptide or protein is different from the second peptide or protein.

4. A method as claimed in claim 3, wherein:
a) the molecular weights of the first and second proteins or peptides are within a 20000 Da, 15000 Da, 10000 Da, 7000 Da, 5000 Da, 4000 Da, 3000 Da, 2500 Da, 2000 Da, 1000 Da, 500 Da, 100 Da, 50 Da, 20 Da or 5 Da range;
b) the molecular weight of the second peptide or protein is within a range of ±50%, ±30%, ±25%, ±20%, ±15%, ±10%, ±5%, ±2% or ±1% of the molecular weight of the first peptide or protein; and/or
c) the first protein or peptide corresponds to an isolated fragment of the second protein or peptide.

5. A method as claimed in claim 3, wherein the first and second peptides or proteins:
a) have an isoelectric point within a 5, 4, 3, 2 or 1 unit range; and/or
b) have at least 50%, 60%, 70%, 80° A, 90%, 95° A, 97° A, 98% or 99% sequence identity.

6. A method as claimed in claim 1, wherein the first and second peptides or proteins are both water-soluble.

7. A method as claimed in claim 1, wherein the molecularly imprinted polymer is imprinted with the first peptide or protein in the solution phase.

8. A method as claimed in claim 1, wherein the molecularly imprinted polymer is an organic polymer.

9. A method as claimed in claim 8, wherein the molecularly imprinted polymer is a polymer formed from a monomer(s) having the formula: 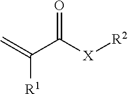
wherein R¹ is H or alkyl; X is O or NR³; R³ is H or alkyl; and R² is H, alkyl, aryl, alkaryl or arylalkyl, each of which may be optionally substituted by one or more hydroxyl, ether, carboxylic acid or ester, amine, amide, imide, sulphonic acid or ester, halogen, or nitrile groups.

10. A method as claimed in claim 1, wherein the molecularly imprinted polymer is a polymer of one or more of the following monomers: acrylamide, an α-alkyl-acrylamide, an N-substituted- or N,N-disubstituted-acrylamide, an N-substituted- or N,N-disubstituted-α-alkyl-acrylamide, acrylic acid, an α-alkyl-acrylic acid, an acrylate, or an α-alkyl-acrylate.

11. A method as claimed in claim 10, wherein the molecularly imprinted polymer is cross-linked using a cross-linking component, the molar ratio of the monomer component to the cross-linking component being in the range 10:1 to 30:1, 15:1 to 25:1, or 18:1 to 22:1.

12. A method as claimed in claim 1, wherein the molecularly imprinted polymer is an inorganic polymer.

13. A method as claimed in claim 12, wherein the molecularly imprinted polymer is formed from a silicon-based monomer.

14. A method as claimed in claim 13, wherein the molecularly imprinted polymer is formed from the following monomer: aminopropyl triethoxysilane (APES).

15. A method as claimed in claim 1, wherein the molecularly imprinted polymer is in gel, or thin film form or sol-gel form.

16. A method as claimed in claim 1, comprising growing a crystal of said second peptide or protein on said molecularly imprinted polymer.

17. A method of purifying a protein or peptide, comprising crystallizing said protein or peptide using a method as claimed in claim 16.

18. A crystal obtainable by a method as claimed in claim 16.

19. The use of a molecularly imprinted polymer to form a nucleus of, and/or to crystallize, a peptide or protein.

20. The use of claim 19, wherein the molecularly imprinted polymer is used in a high-throughput or automated mode.

21. A method for screening for appropriate protein or peptide crystallization conditions, comprising:
providing a molecularly imprinted polymer imprinted with a first protein or peptide;
conducting pairs of screening trials for a second protein or peptide, each pair relating to atrial under a different one of a plurality of different sets of conditions, one member of each pair including the molecularly imprinted polymer in a solution of the second protein or peptide and the corresponding other member of the pair including no molecularly imprinted polymer; and:
a) determining the conditions under which nucleation of the second protein or peptide occurs in the trials that include the molecularly imprinted polymer, but under which no nucleation occurs in the corresponding trials including no molecularly imprinted polymer, thereby identifying metastable conditions for the second protein or peptide; and/or
b) determining the conditions under which nucleation of the second protein or peptide occurs in the trials that include no molecularly imprinted polymer, but under which nucleation occurs at a faster rate in the corresponding trials including the molecularly imprinted polymer, thereby identifying nucleation conditions for the second protein or peptide.
